(19) United States
(12) Patent Application Publication
(10) Pub. No.: US 2009/0105811 A1
(43) Pub. Date: Apr. 23, 2009

(54) Intravascular Devices for Cell-Based Therapies

(75) Inventors: Thomas Q. Dinh, Minnetonka, MN (US); Rodolfo A. Padua, Richfield, MN (US); Syamasundar V. Pakala, Blaine, MN (US); Matthew Jolly, Minneapolis, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/254,258

(22) Filed: Oct. 20, 2008

Related U.S. Application Data
(60) Provisional application No. 60/981,036, filed on Oct. 18, 2007.

(51) Int. Cl. A61F 2/82

(52) U.S. Cl. 623/1.41

(57) ABSTRACT

Disclosed herein are vascular cell encapsulation devices that can deliver cell type(s) for the treatment of a disease or disorder.

### FIELD OF THE INVENTION

The present invention provides cell encapsulation devices that can be implanted into the vascular system to treat a variety of diseases and disorders caused by malfunctioning cell type(s).

### BACKGROUND OF THE INVENTION

The treatment of a number of diseases or disorders could be assisted by the implantation of functioning cell types that are otherwise defective in a patient population. For example, many diabetic patients have malfunctioning insulin-producing cells. Thus, it has long been recognized that restoring normal insulin-producing cell function would be a dramatic treatment breakthrough. There have been several approaches to this end.

First, because insulin-producing cells are located in the pancreas, one early approach was to transplant human or animal pancreatic tissue into diabetic patients. This approach can be problematic, however, because the transplanted tissue is often recognized by the immune system as foreign and destroyed. To address this problem, transplant patients are often placed on immunosuppressive drugs, making them more susceptible to infection. Furthermore, other problems can be caused by molecules other than insulin that are produced by the implanted pancreatic tissue. For example, in addition to insulin, the pancreas produces a variety of enzymes that, when released, interfere with healing after the transplant.

Another approach to restoring insulin-producing cell function has been to enclose insulin-producing cells within a semi-permeable membrane and then implant the encapsulated cells into the body. This approach has several advantages. First, when an appropriate pore size on the semi-permeable membrane is chosen, it is possible for oxygen and glucose to diffuse through the membrane and directly interact with the insulin-producing cells to stimulate natural and timely insulin release. This pore size can also prevent immune system cells from reaching the insulin-producing cells, thus helping to prevent their destruction. Moreover, because only insulin-producing cells are included within the membrane, the problematic release of other enzymes does not occur. These devices, often called cell encapsulation devices (CEDs), theoretically allow the implanted insulin-producing cells to release insulin in real time according to changing glucose levels in the blood.

Unfortunately, while the described CEDs provided some hope for cell replacement therapies, there were key drawbacks associated with their use that prevented them from providing viable treatment options. For example, many previously used CEDs were implanted in the subcutaneous (under the skin) space. Exposure to oxygen, therefore, a condition necessary for cell survival, was dependent on the presence of small capillaries in the area. The location of capillaries, however, can be extremely fickle. Moreover, even when present, these small arteries often did not carry enough oxygen to sustain the viability of the encapsulated cells.

To address these issues related to low oxygen availability in the subcutaneous space, CEDs were developed that could be exposed to a higher volume of blood flow (and hence more oxygen) by inserting them in or attaching them to a blood vessel directly. These CEDs, however, also encountered problems that prevented them from providing viable treatment options. For example, many of them significantly disrupted blood flow, a medically impermissible outcome. Others required invasive and dangerous implantation procedures that rendered them clinically nonviable. Therefore, to date, there is still a need for a device that can provide sustainable functioning cell types to patients suffering from a disease or disorder caused by malfunctioning cell type(s).

### SUMMARY OF THE INVENTION

The present invention addresses drawbacks associated with the prior art by providing a cell encapsulation device (CED) that can be inserted into the vascular system with minimally invasive procedures. Moreover, the presently disclosed devices do not significantly disrupt blood flow. These devices, then, provide a mechanism to provide functioning cell types to a patient wherein the implanted cells have a sufficient oxygen supply to promote survival—that is, the present invention provides a true vascular cell encapsulation device (VCED).

The present invention achieves these advances in the art by providing, in one example, a tubular structure that will not significantly affect blood flow such as, without limitation, a vascular stent or stent graft. The tubular structure will have (and therefore will define) at least one void that is open to its inner surface with a semi-permeable material placed over the portion of the at least one void that is open to the inner surface. Cells can be loaded into the void(s) that are open to the tubular structure's inner surface with the cells held in place by the inner surface material and a second barrier.

In some embodiments, the at least one void will also be open to the tubular structure's outer surface and a material will also be placed over the portion of the void that is open to the outer surface (lumen wall-facing). Cells can then be loaded into the void space(s) of the tubular structure found between the inner surface material and the other surface material. In this example, cells can be loaded into the voids before or after device implantation. In another example where voids are open to both the inner and outer surfaces of a tubular structure, no material is placed over the outer surface. In this example, once the device is implanted, the blood vessel wall provides the second barrier to keep the cells within the voids. In this example, cells are loaded post-device implantation. Importantly, in these examples, at least the portion of the material on the inner surface covering voids open to the inner surface will be semi-permeable to allow the passage of oxygen and other molecules while preventing the passage of immune system cells that might otherwise attack the implanted cells. In preferred embodiments, the whole material on the inner surface of the structure will be semi-permeable.

In another example of the VCEDs of the present invention, small hollow tubes made at least in part of a semi-permeable material are attached to a central scaffold and cells can be loaded into the tubes. Because the tubular structures and central scaffolds of the present invention comprises devices that are already used or are similar to those presently used in the maintenance of blood flow, the devices of the present invention do not impermissibly impede blood flow and can be implanted with currently practiced, minimally-invasive procedures. Therefore, this approach addresses the key drawbacks associated with previously used CEDs providing a true VCED that allows the effective implantation of sustainable functioning cell types in patients.

Specifically, one embodiment according to the present invention includes a vascular cell encapsulation device (VCED) comprising a tubular structure wherein the tubular structure comprises an inner surface and an outer surface, and wherein the tubular structure defines at least one void that is open to the inner surface and wherein the VCED further comprises an inner surface material located over the at least one void open to the inner surface wherein at least a portion of the inner surface material over the at least one void is semi-permeable and wherein cells can be held within the at least one void between the inner surface material and a second barrier. In another embodiment according to the present invention, the at least one void defined by the tubular structure is also open to the outer surface and the second barrier is an outer surface material located over the portion of the void open to the outer surface or when the VCED is implanted in a patient, the second barrier is created by a blood vessel wall.

In another embodiment according to the present invention, the VCED comprises a central scaffold which supports at least one hollow tube wherein at least a portion of the at least one hollow tube comprises a semi-permeable material and wherein the at least one hollow tube can hold cells. In another embodiment including hollow tubes on a central scaffold, the at least one hollow tube comprises an outer surface which defines one or more voids on at least a portion of the hollow tube surface, a semi-permeable material overlays at least the portion of the hollow tube outer surface having the one or more voids, and the VCED is adapted to hold additional cells of the same type or a different type than those found within the at least one hollow tube between the outer surface of the at least one hollow tube and the interior surface of the overlaying semi-permeable material.

In certain embodiments according to the present invention, the chosen tubular structure or central scaffold can be selected from the group consisting of a stent, a stent graft, and a vascular graft. In particular embodiments, the tubular structure or central scaffold will be a stent.

The voids used in accordance with the present invention can include primary voids, secondary voids, or a combination of primary and secondary voids as described below.

In various embodiments according to the present invention the chosen semi-permeable material will be a semi-permeable membrane or a cross-linked hydrogel. One appropriate semi-permeable membrane for use with the present invention comprises pores with a molecular weight cut off ranging from about 20,000 daltons to about 200,000 daltons. In a particular embodiment where a hydrogel is used as the semi-permeable material, the hydrogel will be cross-linked.

In one embodiment according to the present invention, the cells that can be loaded into the described VCEDs are insulin-producing cells. In another embodiment, the insulin-producing cells are selected from the group consisting of pancreatic cells, islet pancreatic cells, clusters of islet pancreatic cells, genetically-engineered insulin-producing cells and combinations thereof. Cells loaded into the VCEDs of the present invention can also be within a hydrogel matrix.

Vascular cell encapsulation devices according to the present invention can also further comprise at least one bioactive agent. In certain embodiments the VCED is adapted to release the at least one bioactive agent according to a controlled release schedule. In particular embodiments according to the present invention the bioactive agent can be selected from the group consisting of rapamycin, FK506, azathiorprine, mycophenolic acid, vascular endothelial growth factor (VEGF), basic fibroblast growth factor (bFGF), and combinations thereof. The described bioactive agents can be coated onto the tubular structures or central scaffolds described above, can be found within voids or hollow tubes, and/or can be coated onto the interior and/or exterior surface of the at least one hollow tube, the semi-permeable material or a secondary barrier.

The present invention also includes methods. In one embodiment the method includes treating a subject by implanting any one of the VCEDs described above at a medical facility wherein before the subject leaves the medical facility following the implanting, the VCED comprises cells in at least one void and the VCED is contacting a bodily fluid of said subject.

The present invention also includes kits. In one embodiment, the kit comprises any of the VCEDs described above. In another embodiment, the kit comprises any of the VCEDs described above and a cell. In another embodiment, the kit comprises any of the VCEDs described above and a vehicle for administration and/or maintenance of a cell. In another embodiment, the kit comprises any of the VCEDs described above and a VCED delivery device. In another embodiment, the kit comprises any of the VCEDs described above and an injection syringe. In another embodiment, the kit comprises any of the VCEDs described above and instructional information.

### BRIEF DESCRIPTION OF THE FIGURES

FIG. 1A depicts one embodiment of a vascular cell encapsulation device (VCED) of the present invention adopting inner and outer surface materials.

FIG. 1B depicts another embodiment of a VCED of the present invention adopting an inner surface material with a blood vessel wall providing the second barrier.

FIGS. 2A and 2B depict another embodiment of a VCED of the present invention that includes secondary voids as described below.

FIGS. 3A-3D depict hollow tube embodiments of VCEDs of the present invention.

### DETAILED DESCRIPTION OF THE INVENTION

A number of diseases and disorders are caused by the malfunctioning of a particular cell type. Types of diabetes, for example, can be caused by the malfunctioning of insulin-producing cells in the pancreas.

One avenue that has been explored to treat diseases and disorders caused by malfunctioning cell type(s) has been the implantation of cell encapsulation devices (CEDs). In these devices, functioning cell types are housed within a semi-permeable membrane having a pore size such that oxygen and other molecules important to cell survival and function can move through it but that cells of the immune system cannot. In the case of diabetes, this approach can allow glucose and oxygen to stimulate insulin-producing cells to release insulin as required by the body in real time while preventing immune system cells from recognizing and destroying the implanted cells as foreign or allowing the implanted cells from escaping encapsulation.

While previously developed CEDs offered theoretical promise, there were significant problems associated with their clinical application. For example, most of these devices were implanted subcutaneously. Exposure to oxygen, therefore, was dependent on the presence of small capillaries in the area. The location of capillaries, however, can change. Moreover, even when present, these small arteries often do not carry enough oxygen to sustain the viability of the encapsulated cells.

To address these issues, CEDs were developed that could be exposed to a higher volume of blood flow (and therefore oxygen) by inserting them in or attaching them to a blood vessel directly. These CEDs, however, also encountered problems that prevented them from providing viable treatment options. For example, many of them significantly disrupted blood flow. Others required invasive and dangerous implantation procedures that rendered them clinically nonviable. Still others required an external port to the surface of the skin leading to risks of infection. Therefore, none of these devices provided an acceptable means to provide a patient with functioning cell types wherein the implanted cells had a sufficient oxygen supply to maintain their viability.

The present invention provides implantable vascular cell encapsulation devices (VCEDs) that address the drawbacks of the prior art. In one example, the VCEDs of the present invention comprise tubular structures having an inner and outer surface wherein the tubular structure defines at least one void and wherein the at least one void is open to the inner surface of the tubular structure. Particularly appropriate tubular structures are vascular stents or stent grafts. The tubular structure has a semi-permeable material placed over at least a portion of the voids that are open to its inner (blood-facing) surface. In preferred embodiments according to the present invention, more than one void that is open to the tubular structure's inner surface will be present and all or a majority of the voids will also be open to the tubular structure's outer surface. In some embodiments including this void type that is open to both inner and outer surfaces, a second material can be placed over at least a portion of the voids on the tubular structure's outer surface. If an outer surface material is not provided over one or more of this type of void, a blood vessel wall can provide the outer surface barrier for cell encapsulation following implantation.

As should be understood by one of ordinary skill in the art, when a material is only placed on a tubular structure's inner surface, and when the voids are not open to the tubular structure's outer surface, cells can be loaded into the voids found between the inner surface material and the material making up the tubular structure. When a material is only placed on a tubular structure's inner surface, and when voids are open to the tubular structure's outer surface, cells can be loaded into the voids found between the inner surface material and the blood vessel wall following implantation. When a material is placed on both the inner and outer surfaces of the tubular structure, and voids are open to both inner and outer surfaces, cells can be loaded into the voids before or after device implantation. Various embodiments according to the present invention can adopt different void configuration and cell encapsulation approaches alone or in combination on a particular device. To facilitate vascular implantation with minimally invasive procedures, the VCEDs of the present invention can include an expandable structural scaffold.

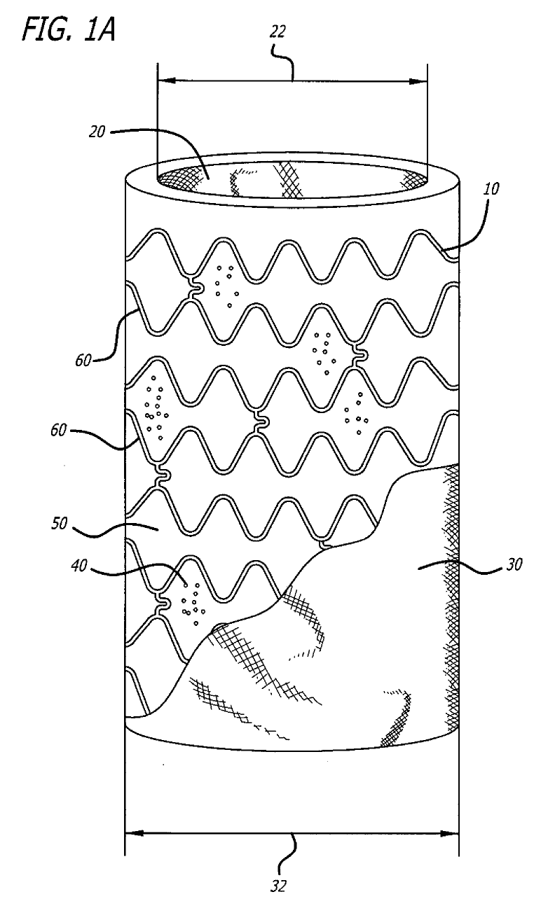

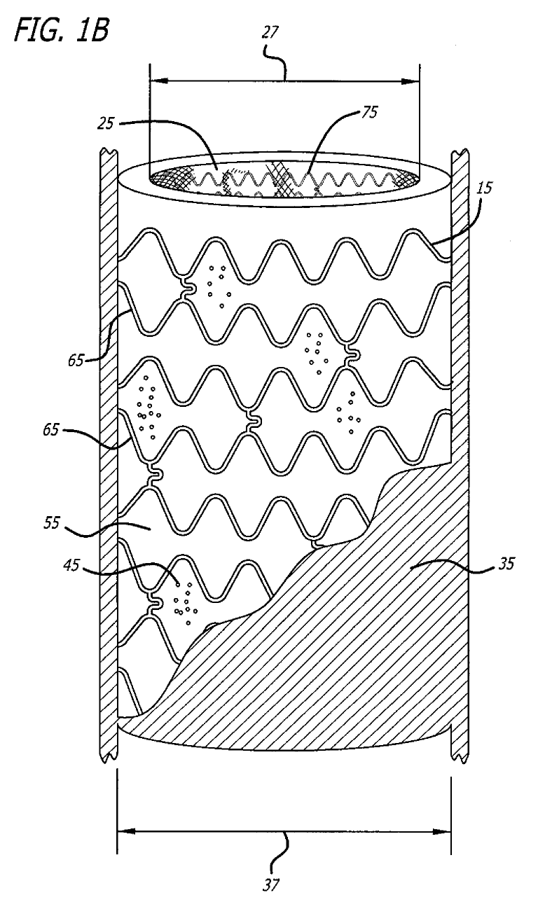

FIG. 1A depicts one particular embodiment of a VCED of the present invention. As can be seen, in this depicted embodiment, the VCED comprises a vascular stent 10 as the central tubular structure. The vascular stent 10 has a material 20 over its inner surface diameter 22 at least a portion of the material 20 being semi-permeable and, in this embodiment, a second material 30 over its outer surface diameter 32. Note, that at least a portion of the material covering voids on the tubular structure's inner surface must be semi-permeable. In preferred embodiments, the entirety of this inner surface material will be semi-permeable. Note also that in many embodiments, the material over the tubular structure's inner and outer surfaces will be the same, and thus, both inner and outer surfaces of the tubular structure will be covered by a semi-permeable material. Cells 40 are loaded in the voids 50 found between the struts 60 of the vascular stent 10. Within the context of the present invention, this type of void space defined by the tubular structure that is open to both the inner and outer surface of the tubular structure is referred to as a “primary void.” Primary voids can also consist of throughbores in a tubular structure. “Voids” as used in accordance with the present invention can also include those described below as “secondary voids” which consist of, without limitation pores or channels created within the wall of a chosen tubular structure. These “secondary voids” are open to either the tubular structure's inner or outer surface, but not both.

FIG. 1B depicts another particular embodiment of a VCED of the present invention implanted in a vessel. In this embodiment the VCED also comprises a vascular stent 15 as the central tubular structure. The vascular stent 15 has a material 25 over its inner surface diameter 27 at least a portion of the material 25 covering voids being semi-permeable. In this embodiment, the VCED does not include a second material over its outer surface diameter 37, but instead the blood vessel wall 35 provides the second barrier to contain the cells 45 within the provided primary voids 55. Again, cells 45 are loaded in the primary voids 55 found between the struts 65 of the vascular stent 15.

This depicted embodiment also includes a stent 75 deployed within the inner surface diameter 27. This stent 75 can be provided in certain embodiments to assist in holding the inner material 25 against the provided tubular structure 15. Stent 75 is not required, however, and inner material 25 can be held in place with appropriate methods known to those of ordinary skill in the art, such as, without limitation, adhesives, suturing, etc.

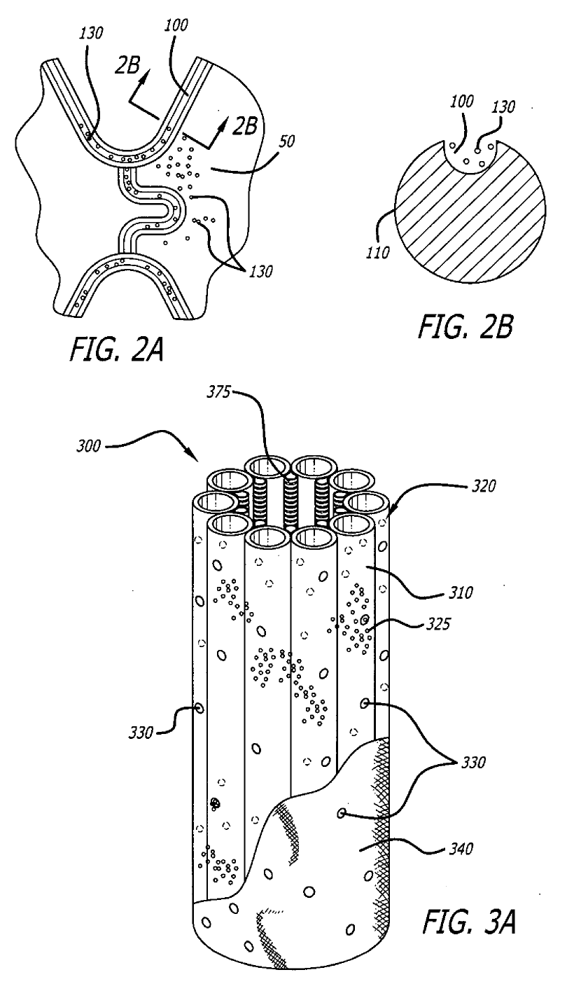

FIGS. 2A and 2B depict an embodiment of the present invention comprising secondary voids 100 created in the struts 110 of a vascular stent. In embodiments comprising secondary voids 100, cells 130 can be loaded into primary voids 50 described as in relation to FIG. 1, into secondary voids 100 or into both types of voids. Moreover, in some embodiments, a chosen tubular structure may include only secondary voids. Secondary voids 100 can be in any suitable form, such as for example, channels, pores, wells, etc. Secondary voids in the forms of channels can be especially beneficial to embodiments of the present invention because this type of secondary void can facilitate cell re-loading of the device as described below following implantation.

When secondary voids 100 are used in accordance with the present invention, in certain embodiments, these secondary voids 100 can be about 100 microns to about 400 microns deep and about 50 microns to about 500 microns in diameter and can be formed in the tubular structure substrate by a variety of methods known to persons of ordinary skill in the art. For example, in one embodiment the secondary voids 100 can be formed using laser/pulse lasers on the blood or lumen-facing side of the tubular structure. While the present size examples are provided, these examples should not be interpreted to limit the scope of the present invention and the types and sizes of secondary voids encompassed thereby.

As stated, certain embodiments according to the present invention comprise a stent, stent graft or vascular graft as the tubular structure. Non-limiting exemplary structures suitable for use in these embodiments are disclosed in U.S. Pat. Nos. 6,812,217; 6,383,214; 6,355,063; 6,312,462; 6,245,099; and 6,124,523, the contents of which are incorporated by reference herein for all they contain regarding intravascular devices. Furthermore, and as will be understood by one of ordinary skill in the art, when a stent, stent graft or vascular graft is used as the tubular structure, these structures can be manufactured in a variety of lengths and diameters from a variety of materials ranging from metallic materials to biocompatible polymers. Metals suitable for fabricating stents for use with the present invention include, without limitation, stainless steel, tantalum, titanium, nickel-titanium alloys, magnesium, shape memory alloys, super elastic alloys, low-modulus Ti—Nb—Zr alloys, and cobalt-nickel alloy steel (MP-35N).

In another embodiment of the present invention, the VCEDs of the present invention include one or more hollow tubes, preferably supported by a central scaffold. The central scaffold can, but need not, be a stent or stent graft. The hollow tubes are preferably a material at least a portion of which is semi-permeable. In preferred embodiments the entirety of the material creating the hollow tubes will be semi-permeable. An additional exemplary appropriate hollow tube structure is disclosed in a co-pending U.S. patent application Ser. No. 11/780,702 filed Jul. 20, 2007. This application is incorporated by reference for all it contains regarding hollow tube structures.

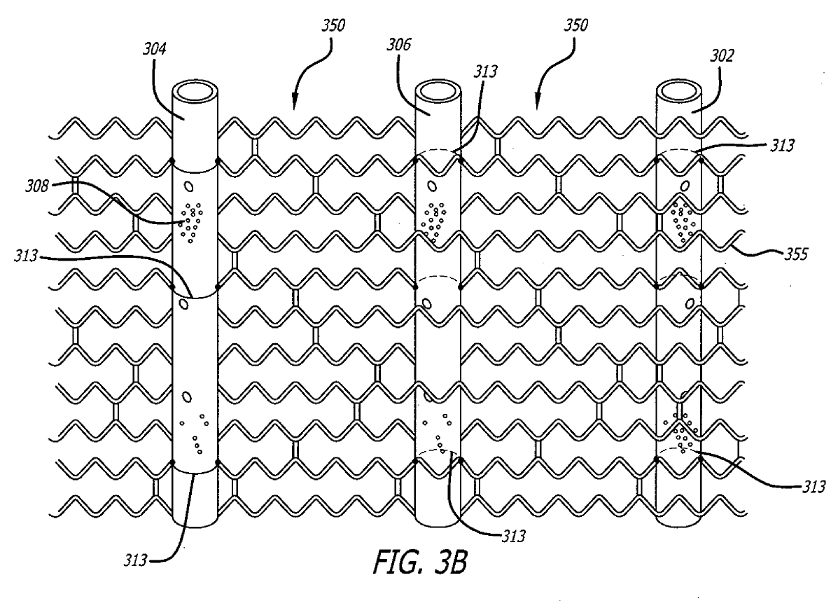

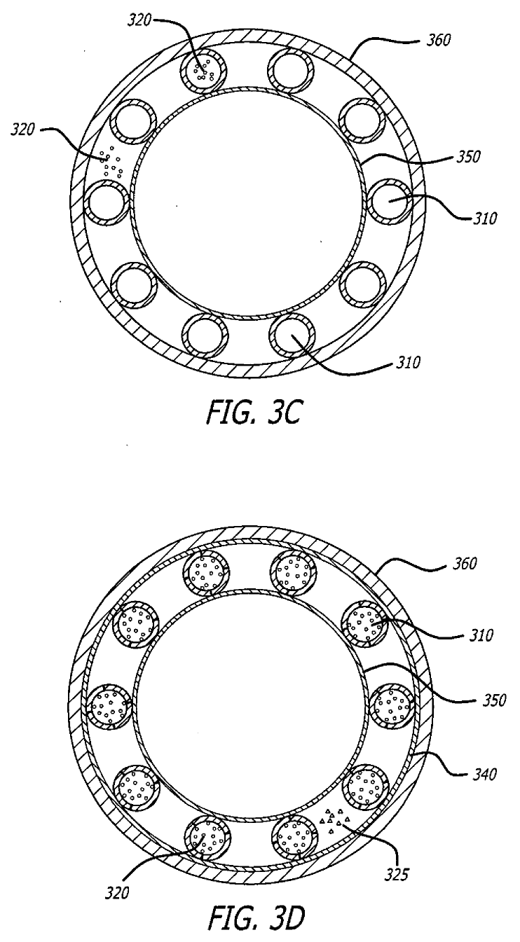

One hollow tube structure embodiment of the present invention is depicted in perspective in FIG. 3A. In this depicted embodiment, the tubular structure 300 comprises a plurality of generally elongate tubes 310, such as hollow fibers. Note, however, that one of ordinary skill in the art will realize that these hollow tubes can come in a variety of lengths, shapes, sizes and diameters. Moreover, the tubes need not be entirely “hollow” but rather must only have a cavity within which cells can reside. Thus, the term “hollow” should be interpreted throughout this description as a cavity for holding cells either alone or in an appropriate cellular matrix.

The elongate tubes 310 depicted in FIG. 3A are preferably constructed of the same material as the semi-permeable materials described above. In one embodiment, a first type of cells 320 (such as islets; seen in phantom) are loaded into the hollow tubes 310 (which allow the passage of oxygen and other important molecules while preventing the passage of immune system and other undesirable cells). In another embodiment, the hollow tubes include additional pores 330 that can have cells loaded into them and can further be covered with a semi-permeable material 340 to further encapsulate additional first type of cells 320 or a second type of cell 325 different than the first type, such as, without limitation, sertoli cells, autologous smooth muscle cells, endothelial cells, and genetically engineered autologous cells that secrete bioactive agents.

In the hollow tube embodiment depicted in FIG. 3A, the elongate tubes 310 are supported on a central scaffold 375. Note that in this depicted embodiment the hollow tubes 310 are attached to the outer surface of the central scaffold 375. In various other embodiments, the hollow tubes of the present invention can be attached to the inner surface of a central scaffold, or, when an appropriate central scaffold such as, without limitation, a stent is chosen, can span the inner and outer surfaces of the scaffold, by for example, being intertwined around the struts of the central scaffold stent. FIG. 3B is a representative flat illustration of an embodiment adopting the use of a stent as a central scaffold 350 for hollow tubes wherein the hollow tubes can be connected to the inside surface of the stent (tube 302), the outside surface of the stent (tube 304) or can be intertwined through the struts of the stent 350 (tube 306). These tubes can be connected to the central stent 350 at one or more attachment points 313 along their length as appropriate. Any attachment method known to those of ordinary skill in the art can be used.

In certain embodiments, the hollow tubes 310 can contact each other as is shown in FIG. 3A (whether or not using a scaffold 375) or can be dispersed around a scaffold 350 as is shown in FIGS. 3B, 3C and 3D. The tubes not touching each other, such as depicted in FIG. 3C, provides a preferred embodiment as more surface area of the tubes is available to contact and interact with passing blood flow. FIG. 3C shows a cross-section of an embodiment of the invention utilizing tubes 310 located on a central scaffold 350. The tubes are adapted to hold cells 320 and to contact the inner wall of vessel 360. Note again that in this depicted embodiment cells of a same or different type can be loaded into spaces between the tubes following implantation. FIG. 3D shows a cross-section of an embodiment of the invention as shown in FIG. 3C utilizing an additional secondary barrier material 340 and second cell type 325 in addition to first cell type 320. In further certain embodiments, a porous polymeric material, a hydrogel, or a mesh can also be applied over the semi-permeable material.

In certain embodiments of the VCEDs of the present invention, the encapsulated cells include insulin-producing cells for the treatment of diabetes. The insulin-producing cells can be pancreatic cells, pancreatic islet cells, pancreatic islet cell clusters and/or genetically-engineered insulin-producing cells. When pancreatic cells are used as the insulin-producing cells, these cells can come from any suitable species, including, without limitation, human, pig, or dog. These insulin-producing cells can be isolated by any suitable procedure known to those of ordinary skill in the art. In certain embodiments using a genetically engineered cell, the insulin-producing cell can be engineered to express one or more genes selected from the group consisting of growth factors, interleukin 4, manganese superoxide dismutase, and Bcl-2.

One particular non-limiting example of a VCED of the present invention used to treat diabetes includes a femoral artery VCED with an inner diameter (ID) of about 6 mm, an outer diameter (OD) of about 6.8 mm and a length of about 18 mm. In this example, the volume of the cell encapsulation chamber is about 0.144 ml. When used to encapsulate and implant insulin-producing cells for the treatment of diabetes, and assuming the volume of an islet cell cluster to be about 160 um (about 2.14×10−6 ml), this exemplary device can contain about 67,289 islet cell clusters. At 50% packing, this is equivalent to 33,644 islet cell clusters for a femoral artery device.

Another particular non-limiting example of the present invention includes an abdominal aorta VCED. Assuming an ID of about 2.5 cm, an OD of about 2.6 cm and a length of about 3 cm, the volume of this device would be about 1.2 ml. For an islet cell cluster with a volume of about 2.14×10⁻⁶ ml, this device can contain about 560,747 islet cell clusters. At 50% packing, this would be equivalent to about 280,373 islet cell clusters for an abdominal aorta device.

On average, when the VCEDs of the present invention are used to treat diabetes, an appropriate amount of insulin-producing cells will be roughly about 20,000 to about 2 million cells. When using islet cell clusters, use of roughly about 5,000 to about 7,000 islet cell clusters/kg should be appropriate. For purposes of the present invention an islet cell cluster should be interpreted to mean about 1,000 to about 1,500 islet cells and/or islet cell equivalents or a grouping of islet cells and/or islet cell equivalents from about 100 to about 150 microns in size.

The VCEDs described above that are appropriate for use in the femoral artery or the abdominal aorta provide two particular examples of VCEDs of the present invention. Note, however, that the VCEDs of the present invention can be implanted into any blood vessel having sufficient oxygenation, blood flow and diameter to support the device and survival of the encapsulated cells. Thus, in addition to the femoral artery and the abdominal aorta, other particularly appropriate vessels include the subclavian artery and vein. The subclavian vein (which drains the arm) is particularly appropriate because of its easy access based on its closeness to the surface of the skin and its high flow rate, which makes clotting of the blood unlikely. In addition, if clotting should occur, other veins in the area can usually provide adequate drainage of the arm, thus minimizing risk to the patient.

As will be understood by those of ordinary skill in the art, in addition to the treatment of diabetes, the VCEDs of the present invention may be used as a substitute for any selected organ by the use of the appropriate cell type(s). For example, other hormone secreting cells such as parathyroid hormone-secreting cells could be encapsulated and implanted for the treatment of hypoparathyroidism. Hepatocytes could also be encapsulated and implanted for the treatment of patients with liver disease. Chromaffin cells could also be used in the VCEDs of the present invention.

The VCEDs of the present invention can be delivered with minimally-invasive procedures, such as those currently employed to implant vascular stents or stent grafts. In these procedures, the VCED is crimped onto an angioplasty balloon, deployed to the appropriate vascular site and expanded within the vessel. Note that in some embodiments according to the present invention, the VCED will be pre-loaded before implantation with a cell type. In other embodiments, the VCEDs will be loaded post-implantation through the use of a vascular access catheter having a needle that can penetrate the semi-permeable material to introduce the cells into the VCED. Loading cells after a time period following implantation can also be beneficial when it is determined that additional cells may be needed based on treatment effectiveness or based on scheduled re-filling of the device. These additional cells can also be added using a vascular access catheter having a needle.

The semi-permeable materials used in accordance with the present invention can be, without limitation, conventional commercially-available porous membranes or hydrogels. One way to distinguish these two types of semi-permeable materials is to consider relevant molecules either passing through an appropriate pore size found within a conventional commercially-available membrane or by diffusing through a hydrogel. This distinction is not important to the functioning of the disclosed VCEDs, however, and will not be discussed further. In certain embodiments using hydrogels, especially those using a hydrogel as an inner surface material, cross-linking the hydrogel can be beneficial. In other embodiments non-cross-linked hydrogels can be used as a matrix to hold loaded cell types. In other certain embodiments, bioabsorbable or bioerodable membranes can also be used.

The pore size used to create semi-permeable membranes in accordance with the present invention can be of any size that will allow the passage of macromolecules while at the same time preventing passage of cells such as the encapsulated cells out of the device or the passage of immune cells into the device. In certain embodiments, this pore size will correspond to a molecular weight cut off (MWCO) ranging from about 20,000 Daltons to about 200,000 Daltons. Particularly appropriate tissue compatible materials with these characteristics include, without limitation, a copolymer of polyacrylonitrile (for example AN 698 developed by Hospal, France), polysulfone, polyvinylchloride, porous tetrafluoroethylene or a copolymer of any of the aforementioned polymers.

Other polymers useful in the manufacture of suitable semi-permeable materials of the present invention include gelatin, fibrin, collagen, elastin, hyaluronic acid, chondroitin sulfate, dermatan sulfate, heparin sulfate, heparin, cellulose, chitin, chitosan, polyvinylidene fluoride, polyurethane isocyanate, alginate, cellulose and cellulose derivatives (for example, cellulose acetate, cellulose diacetate, cellulose triacetate, cellulose nitrate, cellulose acetate butyrate, ethyl cellulose), polyarylate, polycarbonate, polystyrene, polyimide, polymethylmethacrylate, polyurethane, polyethylene, polyethylene glycol, polypropylene, polyamide, polyester, polycarboxylic acids, polyvinylpyrrolidone (PVP), maleic anhydride polymers, polyamides, polyvinyl alcohols (PVA), polyethylene oxides, polyacrylic acid polymers, polytetrafluoroethylene (PTFE), expanded polytetrafluoroethylene (ePTFE), polyhydroxyethylmethacrylic acid (pHEMA), polyaminopropylmethacrylamide (pAPMA), polyacrylamido-2-methylpropanesulfonic acid (pAMPS), polyacrylamide, polyacrylic acid, mixtures or copolymers thereof, or a wide variety of others typically disclosed as being useful in implantable medical devices. In materials which do not include pores in their usual structural configurations, pores can be introduced by conventional means known by those of ordinary skill in the art.

The materials chosen to overlay appropriate portions of the inner and/or outer surfaces of a chosen tubular structure (and any additional layers placed over these materials) or the hollow tubes disclosed herein can be secured to the tubular structure (or the most closely associated layer in the case of additional layers) by any appropriate securement method generally known to those of ordinary skill in the art. For example, appropriate securement methods can include, without limitation, adhesives, glues, micro-sutures, heat application, etc. Holding these materials in place with a stent as depicted in FIG. 1B is also appropriate.

Various embodiments according to the present invention can further include one or more bioactive agents coated onto all or parts of the provided semi-permeable materials and/or the central tubular structure or scaffold so long as inclusion of these bioactive agents does not significantly interfere with appropriate diffusion through the semi-permeable material. For example, the VCED can be partially or otherwise appropriately coated with a thin layer of polymer containing a bioactive agent.

As used herein, “bioactive agent” shall include any compound or drug having a therapeutic effect in an animal wherein the bioactive agent is not released from the cell types found within a particular VCED. Exemplary, non limiting examples include anti-thrombotic agents, anti-proliferatives, antibiotics, anti-inflammatories, anti-sense nucleotides and transforming nucleic acids. Bioactive agents can also include, cytostatic compounds, toxic compounds, anti-inflammatory compounds, chemotherapeutic agents, analgesics, protease inhibitors, statins, nucleic acids, polypeptides, immune modulators, anti-rejection agents, hormones, immunosuppressive agents, steroidal agents, growth factors (including, without limitation, vascular endothelial growth factor (VEGF) and basic fibroblast growth factor (bFGF)) and delivery vectors including recombinant micro-organisms, liposomes, and the like. Particular non-limiting examples of bioactive agents include rapamycin, FK506, azathiorprine and mycophenolic acid.

In one particular embodiment, the bioactive agent can be an immunosuppressant to further protect the encapsulated cells from unwanted immune system activity. In another particular embodiment the bioactive agent can be an anti-inflammatory drug. In another particular embodiment, the bioactive agent can be an agent that assists in the growth and maintenance of the encapsulated cells in the VCED. Non-limiting examples of such agents includes, but are not limited to, growth factors. As will be understood by those of ordinary skill in the art, combinations of bioactive agents can also be employed. Furthermore, and as will be understood by those of ordinary skill in the art, these bioactive agents can be provided in a variety of controlled release schedules including, without limitation, release schedules ranging from about a few days to about a few weeks to about a few months.

The VCEDs of the present invention can be provided in a kit to promote proper implantation and use. For example, the intended use and function of the VCEDs can be explained by instructional information found with or within the kit (see below). Thus, kits according to the present invention can include one or more of the following components in various combinations associated with each other in one or more appropriate packages or containers; (1) one or more VCEDs of the present invention; (2) one or more cell types; (3) one or more vehicles for administration and/or maintenance of the cell types; (4) one or more VCED delivery devices or catheters; (5) one or more cell injection syringes; and (6) instructions for VCED implantation and use.

When a kit is supplied with one or more cell types, the cells can be subjected to appropriate procedures known to those of ordinary skill in the art during storage and packaging to promote their survival and function following implantation. Kits may also include one or more vehicles for administration and/or maintenance of cell types. These kit components can be packaged in separate or joint containers and/or be admixed immediately before use as appropriate.

Biological components included in particular kits of the present invention can be supplied in containers of any sort such that the life of the different components are preserved and are not adsorbed or altered by the materials of the container. For example, sealed glass ampules may contain appropriate cell type administration vehicles. When used, ampules may also consist of any other suitable materials, such as, without limitation, organic polymers, such as, polycarbonate, polystyrene, etc., ceramic, metal or any other material typically employed to hold similar materials. Other containers include, without limitation, test tubes, vials, flasks, bottles, syringes, or the like. Containers may have one or more sterile access ports, such as a bottle having a stopper that can be pierced by an appropriate injection needle. Other containers may have two compartments that are separated by a readily removable membrane that upon removal permits the components to be mixed. Removable membranes may be, without limitation, glass, plastic, rubber, etc.

Kits according to the present invention can also include VCED delivery devices including, in certain embodiments, delivery catheters. As will be understood by one of ordinary skill in the art, these delivery catheters will generally include at least a lumen, guidewire and expandable balloon at the distal end of the lumen for expanding and deploying the VCEDs of the present invention.

As stated earlier, kits of the present invention can also be supplied with instructional materials. Instructions may be printed on paper or other substrate, and/or may be supplied as an electronic-readable medium, such as a floppy disc, CD-ROM, DVD-ROM, Zip disc, videotape, audiotape, flash memory device, etc. Detailed instructions may not be physically associated with the kit; instead, a user may be directed to an internet web site specified by the manufacturer or distributor of the kit, or supplied as electronic mail.

The present invention also includes methods that comprise treating a subject by implanting any one of the VCEDs described above at a medical facility wherein before the subject leaves the medical facility following the implanting, the VCED comprises cells in at least one void and the VCED is contacting a bodily fluid of said subject. Cells can come pre-loaded in a particular VCED, can be provided as part of a kit with a VCED or can be obtained separately from the VCED. If the cells do not come pre-loaded in a particular VCED, they can be loaded before or after device implantation as appropriate with different void types and treatment goals. Thus, as should be understood, the exact implantation route and procedure, cell type, and cell dosage can be determined by the attending physician in view of the patient's condition. Cell dosage amount and implantation site can be adjusted individually to provide and maintain a therapeutic effect. Furthermore, as used herein, medical facility should be interpreted to mean a location where at least one doctor offers medical care.

Unless otherwise indicated, all numbers expressing quantities of ingredients, properties such as molecular weight, reaction conditions, and so forth used in the specification are to be understood as being modified in all instances by the term “about.” Accordingly, unless indicated to the contrary, the numerical parameters set forth in the following specification are approximations that may vary depending upon the desired properties sought to be obtained. Notwithstanding that the numerical ranges and parameters are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used are to be construed to cover both the singular and the plural, unless otherwise indicated herein or clearly contradicted by context. Recitation of ranges of values herein is merely intended to serve as a shorthand method of referring individually to each separate value failing within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended to better illuminate embodiments according to the invention.

Groupings of alternative elements or embodiments according to the invention disclosed herein are not to be construed as limitations. Each group member may be referred to individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Embodiments of this invention are described herein. Of course, variations on those embodiments will become apparent to those of ordinary skill in the art upon reading the foregoing description unless otherwise indicated herein or otherwise clearly contradicted by context.

Furthermore, numerous references have been made to patents and printed publications throughout this specification. Each of the above cited references and printed publications are herein individually incorporated by reference in their entirety.

1. A vascular cell encapsulation device (VCED) comprising a tubular structure wherein said tubular structure comprises an inner surface and an outer surface, and wherein said tubular structure defines at least one void that is open to said inner surface and wherein said VCED further comprises an inner surface material located over said at least one void open to said inner surface wherein at least a portion of said inner surface material over said at least one void is semi-permeable and wherein cells can be held within said at least one void between said inner surface material and a second barrier.

2. A VCED according to claim 1 wherein said at least one void defined by said tubular structure is also open to said outer surface and wherein said second barrier is an outer surface material located over the portion of said void open to said outer surface or wherein when said VCED is implanted in a patient, said second barrier is created by a blood vessel wall.

3. A VCED according to claim 1 wherein said cells are insulin-producing cells.

4. A VCED according to claim 3 wherein said insulin-producing cells are selected from the group consisting of pancreatic cells, islet pancreatic cells, clusters of islet pancreatic cells, genetically-engineered insulin-producing cells and combinations thereof.

5. A VCED according to claim 1 wherein said cells are within a hydrogel matrix.

6. A VCED according to claim 1 wherein said inner surface material comprises a semi-permeable membrane or a cross-linked hydrogel.

7. A VCED according to claim 6 wherein said inner surface material comprises a semi-permeable membrane comprising pores with a molecular weight cut off ranging from about 20,000 daltons to about 200,000 daltons.

8. A VCED according to claim 1 wherein said tubular structure is selected from the group consisting of a stent, a stent graft, and a vascular graft.

9. A VCED according to claim 8 wherein said tubular structure is a stent.

10. A VCED according to claim 2 wherein said voids are primary voids, secondary voids or primary and secondary voids.

11. A VCED according to claim 1 further comprising at least one bioactive agent.

12. A VCED according to claim 11 wherein said VCED is adapted to release said at least one bioactive agent according to a controlled release schedule.

13. A VCED according to claim 11 wherein said bioactive agent is selected from the group consisting of rapamycin, FK506, azathiorprine, mycophenolic acid, vascular endothelial growth factor (VEGF), basic fibroblast growth factor (bFGF), and combinations thereof.

14. A VCED according to claim 1, wherein said tubular structure is a first tubular structure, and further comprising a second tubular structure located coaxially within said first tubular structure, wherein said inner surface material is located between an outer surface of said second tubular structure and said inner surface of said first tubular structure.

15. A vascular cell encapsulation device (VCED) comprising a central scaffold which supports at least one hollow tube wherein at least a portion of said at least one hollow tube comprises a semi-permeable material and wherein the at least one hollow tube is adapted to hold cells.

16. A VCED according to claim 15 wherein said at least one hollow tube comprises an outer surface and wherein said at least one hollow tube defines voids on at least a portion of said outer surface and wherein a semi-permeable material comprising an interior surface overlays at least said portion of said outer surface having voids and wherein the VCED is adapted to hold additional cells of the same type or a different type than those found within said at least one hollow tube between the outer surface of said at least one hollow tube and the interior surface of said overlaying semi-permeable material.

17. (canceled)

18. (canceled)

19. (canceled)

20. (canceled)

21. A VCED according to claim 15 wherein said central scaffold is selected from the group consisting of a stent, a stent graft, and a vascular graft.

22. (canceled)

23. (canceled)

24. (canceled)

25. A method for treating a subject comprising: implanting a VCED according to either of claims 1 or 15 in said subject at a medical facility wherein before said subject leaves said medical facility following said implanting, said VCED comprises cells in said at least one void and said VCED is contacting a bodily fluid of said subject.

26. (canceled)

27. A kit comprising a VCED according to either of claims 1 or 15.

28. (canceled)

29. (canceled)

30. A kit according to claim 27 further comprising a VCED delivery device.

31. (canceled)

32. (canceled)

33. (canceled)

34. (canceled)

35. (canceled)

36. (canceled)

37. (canceled)

38. (canceled)
